(19) United States
(12) Patent Application Publication
(10) Pub. No.: US 2024/0245873 A1
(43) Pub. Date: Jul. 25, 2024

(54) INTEGRATING ACOUSTIC RESONANCE WITH SLEEP APNEA THERAPY

(71) Applicant: Sound Health Systems, Inc., Los Altos, CA (US)

(72) Inventors: Elan Hekier, San Diego, CA (US); Bryant Lin, Los Altos, CA (US); Paramesh Gopi, Los Altos, CA (US); Vivek Mohan, Sunnyvale, CA (US); Peter H. Hwang, Los Altos, CA (US)

(21) Appl. No.: 18/100,836

(22) Filed: Jan. 24, 2023

(51) Int. Cl. A61M 16/00; A61M 16/06

(52) U.S. Cl. CPC A61M 16/0006; A61M 16/024; A61M 16/0633; A61M 2205/3375; A61M 2230/63

(57) ABSTRACT

A system, method and device for integrating the application of acoustic resonance, as correlated with a wearer of the device, with the application of a continuous positive airway pressure system. The aspects disclosed herein are directed to improving CPAP compliance and efficacy. In various embodiments, the acoustic resonance may be applied directly to a wearer's paranasal sinus, or to an air passageway that communicates with an orifice of a person.

### BACKGROUND

Numerous individuals suffer from sleep apnea. Sleep apnea is a potentially serious sleep disorder in which breathing repeatedly stops and starts. There are multiple forms of sleep apnea, such as obstructive sleep apnea, where throat muscles relax during a sleeping phase, central sleep apnea, where a individual's brain fails to send proper signals associated with controlling muscles connected to breathing, and a complex sleep apnea, being a hybrid of the two.

Untreated sleep apnea is associated with an increased risk for heart attacks, strokes and may shorten one's life expectancy. Even in the non-severe forms, one's quality of life may decrease.

In dealing with sleep apnea, a doctor may prescribe a positive airway pressure (PAP) treatment, for example, through a continuous positive airway pressure (CPAP) device.

FIG. 1 illustrates a prior art CPAP mask 100. The CPAP mask 100 includes an inlet 110, a right 120 and left 130 air passage pathway, and a nostril engagement portion 140. The inlet 110 attaches to a machine (not shown), and receives air in a continuous fashion to be provided to the wearer of the CPAP mask 100. The right 120 and left 130 air passages are cylindrical shaped cavities that allow air to be communicated from the inlet 110 to the nostril engagement portion 140. The right 120 and left 130 air passages may be formed of a flexible material to comport to the shape of the wearer's face.

The CPAP mask 100 is specifically designed to deliver a flow of air at a constant pressure. This constant stream of air opens and keeps the upper airway unobstructed during inhalation and exhalation.

The wearer of the CPAP mask 100 places the nostril engagement portion 140 on their nostril portion of their nose. For a variety of reasons, wearing this device is obstructive and may be difficult to sleep with. As such, a certain percentage of wearers fail to keep the CPAP mask 100 on during sleep. It is estimated that nearly 50% of people discontinue wearing the CPAP device.

This is only further exacerbated when a user is experiencing a secondary nasal condition, for example, congestion or other ailments associated with sinus-related issues.

While FIG. 1 shows one specific embodiment, there have been many devices and attempts to make the CPAP device more form fitting and easier to keep on during a sleep period.

As noted above, there is a great need to improve the existing state of CPAP devices, not only increasing the effectiveness of the therapy, but also increasing the compliance rates associated with wearing the devices.

### SUMMARY

An aspect of some embodiments of the invention relates to a method and systems for augmenting a PAP device with acoustic resonance delivery. In one embodiment, the system for providing positive airflow pressure (PAP), the system being couplable to a wearable PAP device, includes an acoustic resonance actuator configured to deliver acoustic resonance; and a processor configured to generate resonance data, the resonance data being received by the acoustic resonance actuator, the resonance data corresponding to a resonant frequency based on a volume of the wearer's paranasal sinus cavity. The acoustic resonance actuator is configured to deliver the acoustic resonance when the wearable PAP device is delivering airflow.

In another embodiment, the system is defined by resonance data being sourced from a facial scan of the wearer's face.

In another embodiment, the system is further defined by the acoustic resonance actuator is integrated in a band worn on the wearer's forehead.

In another embodiment, the band includes bone conduction speakers configured to be worn on an area over the frontal sinus.

In another embodiment, the acoustic resonance actuator is a speaker embedded on a wall of a portion of the PAP device in which air flows to the nostril, a sound producing portion of the speaker being oriented inside a cavity of the PAP device passage.

In another embodiment, the system further includes an inlet attachment unit, coupled to an inlet of the PAP device, and the acoustic resonance actuator being embedded in a housing of the inlet attachment unit.

In another embodiment, the sensor further is defined by detects motion or sound, and after detecting said motion or sound, applying the acoustic resonance to the wearer.

In another embodiment, the system is defined as the volume being approximated by the facial scan.

According to the aspects disclosed herein, in a preferred embodiment, a method for coupling acoustic resonance for a wearer of a positive airflow pressure (PAP) mask is disclosed. This method includes generating resonance data, the resonance data corresponding to a resonant frequency based on a volume of the wearer's paranasal sinus cavity, and delivering the acoustic resonance to the wearer while simultaneously delivering airflow to the wearer.

According to the aspects disclosed herein, in a preferred embodiment, an inlet attachment device for a PAP device is disclosed. The inlet includes a housing with a first end and a second end, the housing forming a substantially hollow portion; the first end being coupled to an inlet portion for the PAP device; the second end being coupled to an output of a flow generator; a speaker embedded in a wall of the housing, the speaker oriented so as to produce sound to the hollow portion; and a processor configured to provide the sound simultaneously when the flow generator is delivering airflow.

### DESCRIPTION OF THE DRAWINGS

The detailed description refers to the following drawings, in which like numerals refer to like items, and in which:

FIG. 1 illustrates a CPAP mask 100 according to a prior art implementation;

FIG. 2 illustrates a system according to exemplary embodiments disclosed herein.

FIG. 3 illustrates a flowchart explaining the operation of the system shown in FIG. 2.

FIG. 4 illustrates an example of a cranial diagram explaining sinus passages employed for determining a resonant frequency.

FIGS. 5 and 6 illustrate an experiment explaining the use of resonant frequencies as applied to a wearer of a band.

FIGS. 7(a)-(d) illustrate crano-facial critical points used for an estimation of a resonant frequency.

FIGS. 8(a)-(c) illustrate a methodology for determining a resonant frequency based on the employment of critical points extracted in FIGS. 7(a)-(d).

FIGS. 9 and 10 illustrate a first embodiment according to aspects disclosed herein.

FIGS. 11 and 12 illustrate a second embodiment according to the aspects disclosed herein.

FIG. 13 illustrates a third embodiment according to aspects disclosed herein.

FIGS. 14(a)-(c) illustrate an exemplary scenario employing the concepts disclosed herein.

### DETAILED DESCRIPTION

The invention is described more fully hereinafter with references to the accompanying drawings, in which exemplary embodiments of the invention are shown. This invention may, however, be embodied in many different forms and should not be construed as limited to the embodiments set forth herein. Rather, these exemplary embodiments are provided so that this disclosure is thorough, and will fully convey the scope of the invention to those skilled in the art. It will be understood that for the purposes of this disclosure, “at least one of each” will be interpreted to mean any combination the enumerated elements following the respective language, including combination of multiples of the enumerated elements. For example, “at least one of X, Y, and Z” will be construed to mean X only, Y only, Z only, or any combination of two or more items X, Y, and Z (e.g. XYZ, XZ, YZ, X). Throughout the drawings and the detailed description, unless otherwise described, the same drawing reference numerals are understood to refer to the same elements, features, and structures. The relative size and depiction of these elements may be exaggerated for clarity, illustration, and convenience.

In this disclosure, CPAP is used interchangeably with PAP (for example, the CPAP mask may also or alternatively be a PAP mask). In addition to the variation of CPAP devices disclosed above, the aspects disclosed herein may be used with auto adjusting and BiLevel positive airway pressure devices.

CPAP machines may benefit from being more effective and allowing compliance at a greater rate. The aspects disclosed herein are directed towards improvements in both.

Additionally, through studies associated with the aspects disclosed herein, other benefits have been realized. The aspects disclosed herein have led to shortening sleep latency. Sleep latency, in the context of evaluating a patients sleep, is defined as the amount of time it takes a patient to sleep.

Further, in addition to sleep latency improvements, the devices disclosed herein may also decrease time awake after initially falling asleep—referred to wakefulness after sleep onset (WASO). For example, an individual may go to bed at 11:30 p.m. and suddenly rouse from slumber at 2:30 a.m. and remain awake until 3:45 a.m., tossing and turning. Employing various of the devices and embodiments discussed herein, may reduce this phenomenon.

In addition to the above, the device discussed herein may improve and increase deep sleep. It may achieve this by decreasing sleep fragmentation from sleep disorder breathing.

The aspects disclosed herein employ acoustic resonance as applied to a CPAP device based on an estimated size of a sinus portion of a user. The aspects disclosed herein may be integrated into the air passage portion (for example, via an inlet), or separately provided directly to a user.

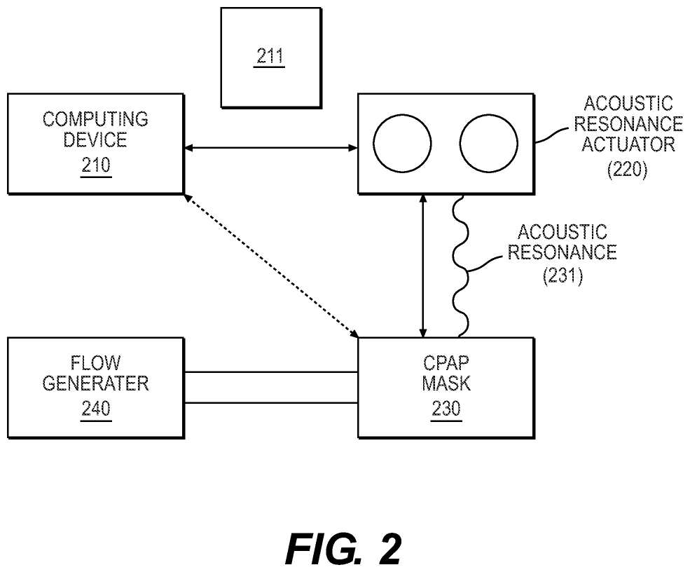

FIG. 2 illustrates an embodiment of the employment of the systems disclosed herein. As shown in FIG. 2, a computing device 210 is coupled to CPAP mask 230. The computing device 210 may be directly coupled to the CPAP mask 230, or to the acoustic resonance actuator 220. According to aspects disclosed herein, the CPAP technology may be implemented with all types of PAP technologies.

Also shown is a flow generator 240 (or a CPAP machine), which is coupled to the CPAP mask 230 via a hose. Flow generator 240 is well known in the art, and a detailed description will be omitted. The computing device 210 may be integrally provided with a flow generator 240, or a processor included or integrated with said flow generator 240. According to the aspects disclosed herein, various portions, may be implemented on multiple computing devices, and in communication with either other through known methods of handshaking multiple devices.

The acoustic resonance actuator 220 generates acoustic resonance 231, through a variety of techniques. In one such preferred embodiment, the acoustic resonance 231 is generated via a bone conducting sound generation system/speaker. Bone conduction speakers can convert an electrical signal into a mechanical vibration signal, and transmit the mechanical vibration signal into a human auditory nerve through human tissues and bones so that a wearer of the speaker can hear the sound.

While the acoustic resonance actuator 220 may be a bone conduction speaker, in other embodiments, the acoustic resonance actuator 220 may be any device capable of producing acoustic resonance 231.

The placement of the resonance actuator 220 may be integrally provided at an inlet of the CPAP mask 230 according to one embodiment disclosed herein, and/or in a separate device also disclosed in an embodiment disclosed herein.

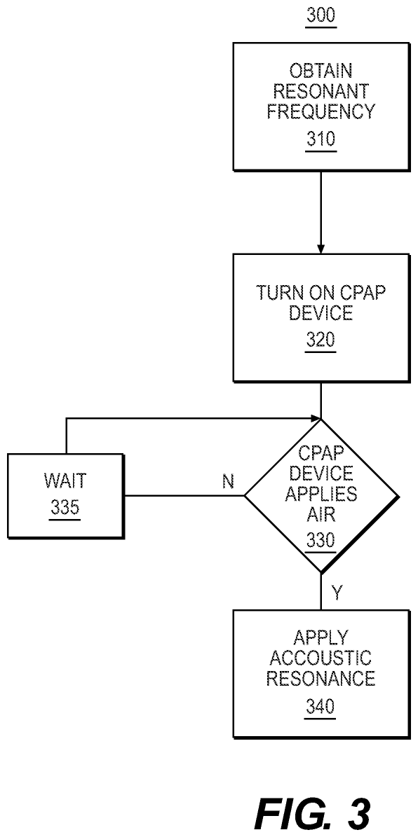

FIG. 3 illustrates a high-level flow diagram 300 explaining an implementation of the computing device 210 according to an exemplary embodiment disclosed herein. The computing device 210 may be any computer or processor (or combination thereof multiple processors) capable of storing and executing instructions.

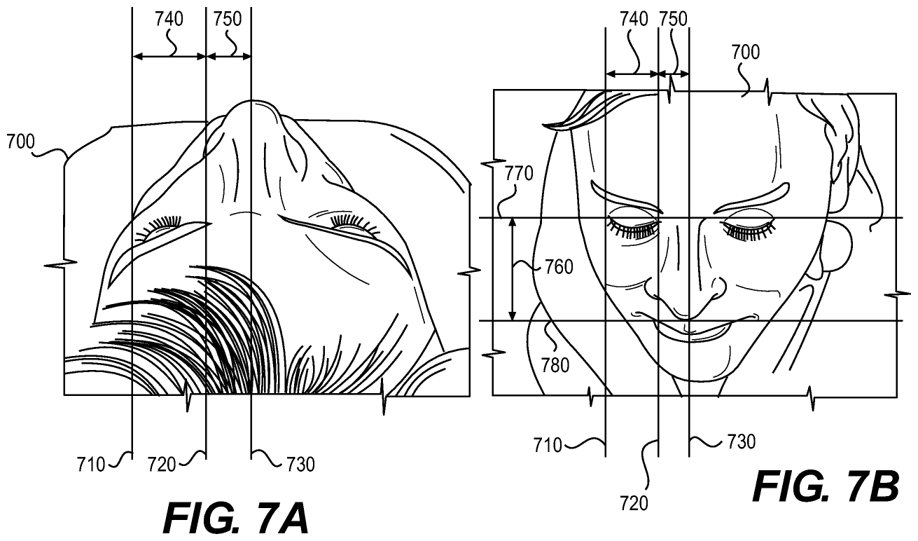

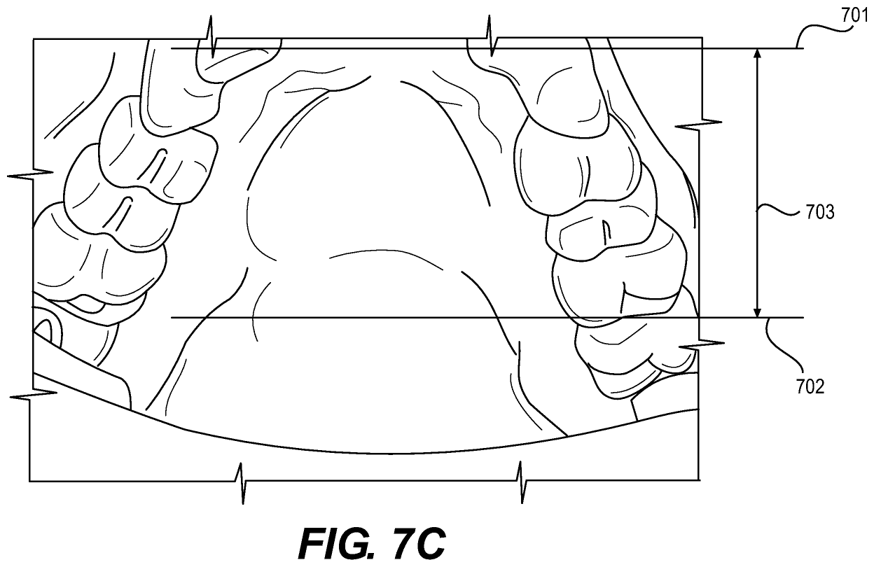

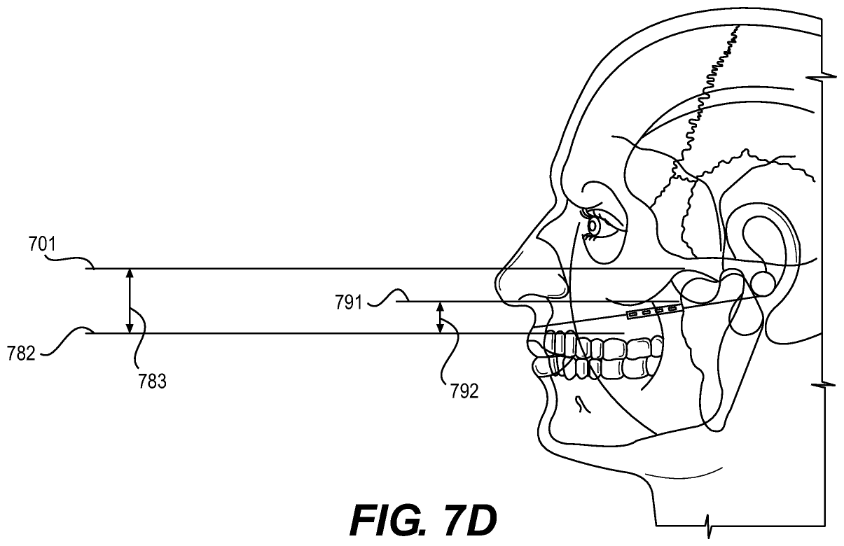

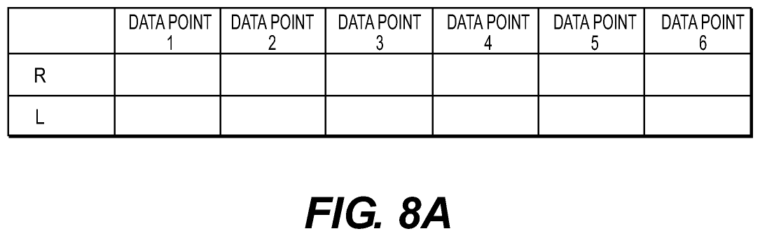

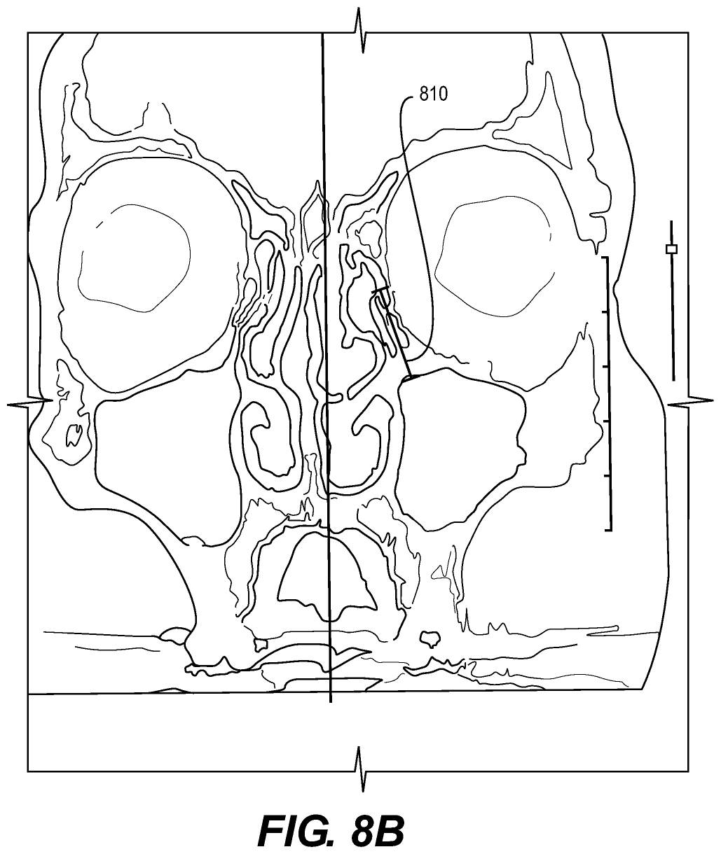

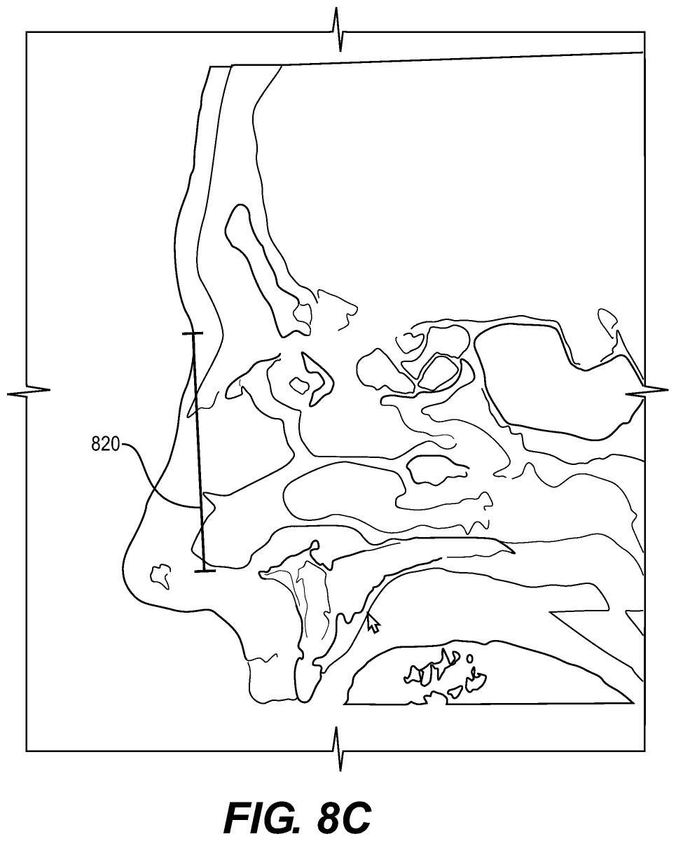

In step 310, a resonant frequency of the wearer of the CPAP mask 230 is obtained. One method of obtaining the resonant frequency is described below (see FIGS. 7(a)-FIG. 8(c)). In this method, a photo of a wearer's face is obtained. And through the photo, the measurement of critical distances on said face from various crano-facial points is obtained, and an acoustic resonant frequency may be produced. The acoustic resonance frequency may be stored as the resonant data 211, and used by the acoustic resonance actuator 230 to produce an acoustic resonance 231.

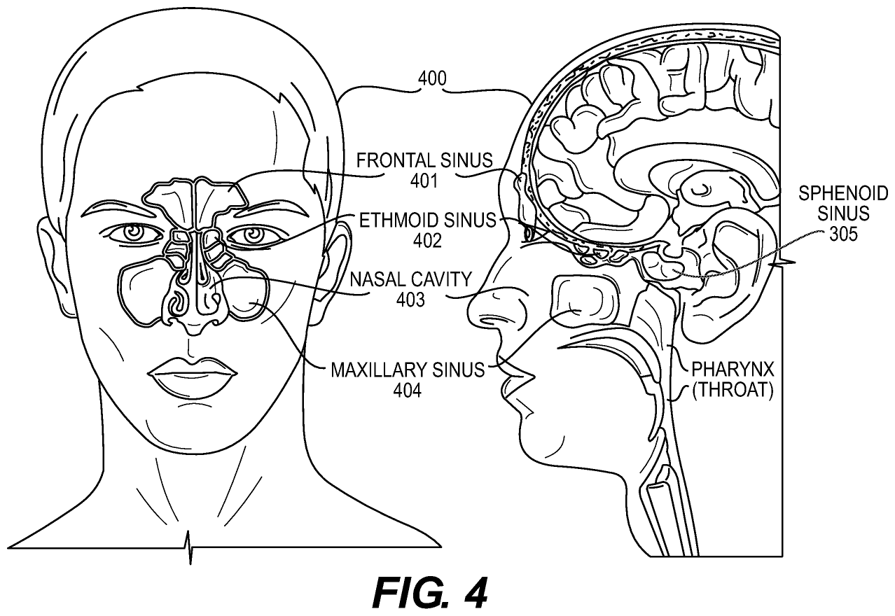

In an example disclosed herein, the resonant frequency 231 corresponds to a calculated or estimated size (i.e. volume) of the sinus passage ways described and shown in FIG. 4.

FIG. 4 illustrates an exemplary head, with various reference points used in determining critical measurements used in step 310. Collectively, the volume of these sinus passageways are estimated/approximated, and used to determine an acoustic resonance 231.

In FIG. 4, a frontal-view and a side-view of an illustration of a head 400 depicting exemplary sinus/nasal tracts on a person. These sinuses are a frontal sinus 401, an ethmoid sinus 402, a nasal cavity sinus 403, and a maxillary sinus 404.

These sinus and nasal tracts may be referred to as paranasal sinuses, and collectively establish critical sinuses that allow access to areas where symptoms associated with inflammation and sinusitis may occur. Paranasal sinuses are a group of four paired air-filled spaces that surround the nasal cavity. The maxillary sinuses 404 are located under the eyes; the frontal sinuses are above the eyes 401; the ethmoidal sinuses 402 are between the eyes and the sphenoidal sinuses 403 are behind the eyes. The sinuses are named for the facial bones in which they are located.

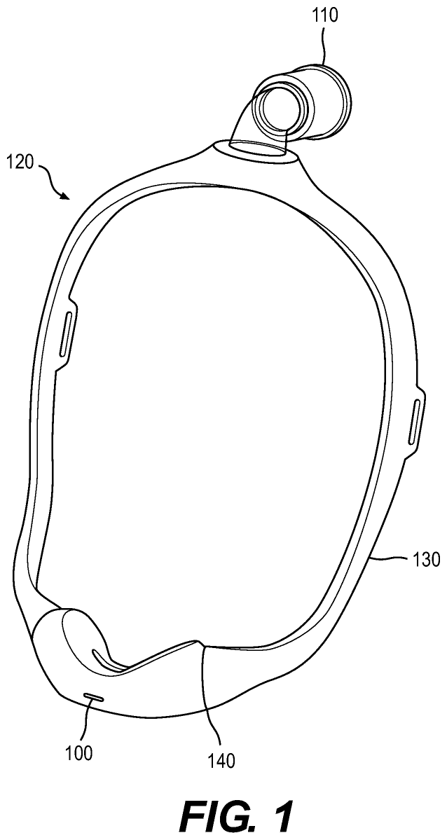

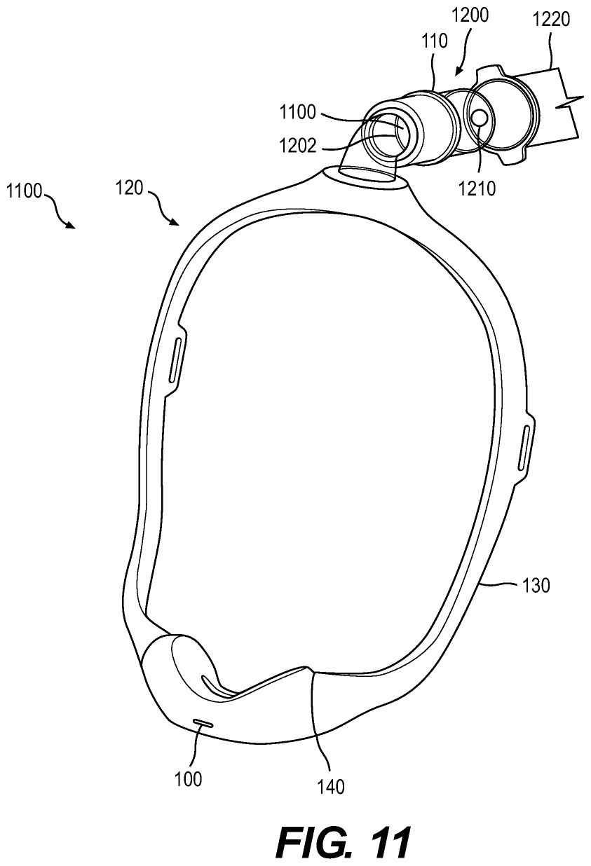

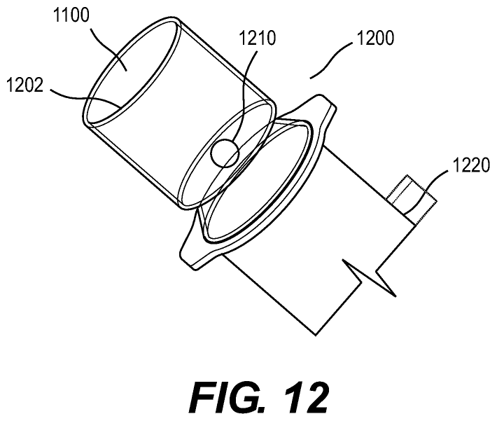

In step 320, the CPAP mask 230 is turned on. The CPAP mask 230 is configured and shaped to be worn by the user. The CPAP mask 230 may be CPAP mask 100 shown in FIG. 1 and provided with an additional device implementing the aspects disclosed herein (as described in FIGS. 11 and 12). Or additionally or alternative to, the CPAP mask 230 may have elements of the aspects disclosed herein built into an existing CPAP mask 100 and integrally provided.

By turning on the CPAP mask 230 in step 320, this enables the CPAP mask 230 to deliver air to the nose or other orifices of the user's body (for example, as shown in FIG. 1, through the nostril). The application of air may be intermittent or continuous and done in a method consistent with known CPAP application techniques.

In step 330, a determination is made as to whether the CPAP mask 230 is in the process of applying air. If no, in one embodiment, a step 335 waiting operation may occur. The waiting operation 335 may then re-instigate the determination in step 330 after a predetermined time or after receiving an on indication from the CPAP mask 230.

In an example discussed below, additional sensors may be included with the CPAP mask 230 or additional devices included to enable the invention. If the sensors detect an instigating motion (for example motion or snoring, or some other biometric sensor), the waiting operation 335 may exit and the method 300 may proceed to step 330/340.

If the answer to step 330 is yes, the method 300 may proceed to step 340, where an acoustic resonance 231 may be applied via the acoustic resonance actuator 220, based on the resonant data 211 created in step 310.

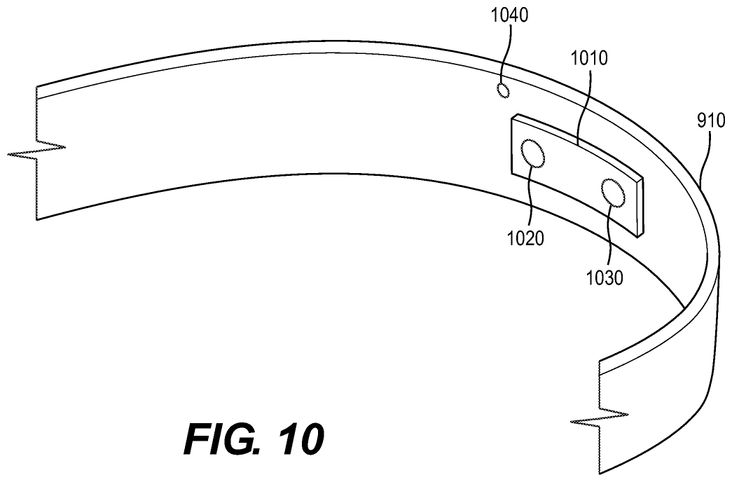

The application of the acoustic resonance 231 may be applied at an inlet (see FIG. 12 for an example) attached to the CPAP mask 230, or via a band worn (see FIG. 10) on the user's forehead. Exemplary versions of both implementations will be described below.

In either case, acoustic resonance 231 is provided to the user while the CPAP mask 230 is operating. The inventors have found through patient studies that the effectiveness of the CPAP mask 230 improves along with the compliance rates associated with said CPAP applications.

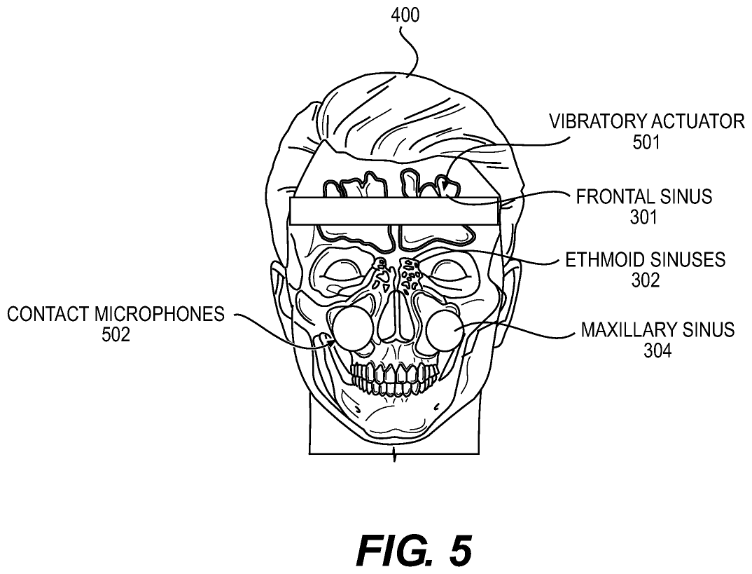

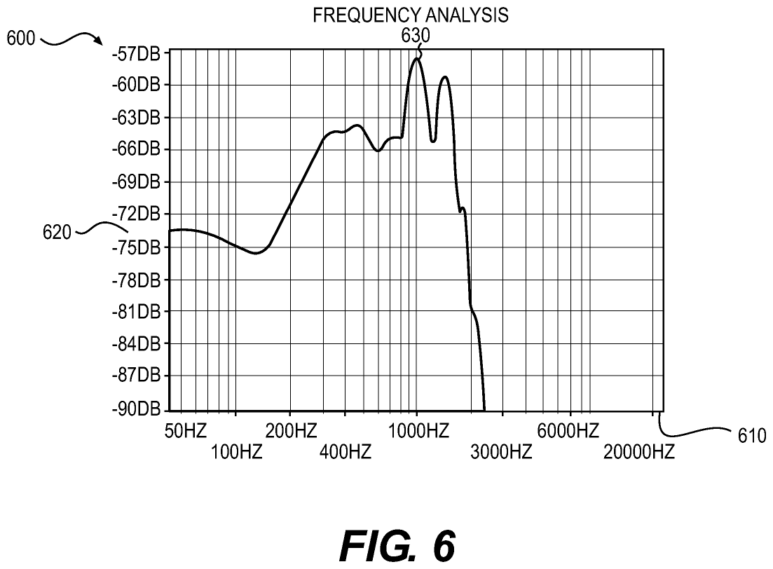

FIGS. 5 and 6 explain the phenomena associated with the application of acoustic resonance 231 to a user.

FIG. 5 is a frontal and side view of the head 400 illustrating an experiment performable with a cadaver. Additionally, to the head 400 shown in FIG. 4, a vibratory actuator 501 is situated over the frontal sinus 401. Also included in FIG. 5 is a contact microphone 502, ideally placed over a maxillary sinus 404.

An experiment was performed utilizing cadavers and the setup in FIG. 5, and as shown in FIG. 6, graph 600 was produced. Graph 600 depicts a spectral analysis of vibratory stimulus to a cadaveric head. On the X-axis 610, various frequencies are swept from a range of 50 Hertz to 3000 Hertz as applied via the vibratory actuator 501. On the Y-axis 620, the sounds generated through the application of a vibratory actuator 501 is captured via the contact microphone 502. Additionally, an air microphone (not shown) may be placed to augment the recording of sound.

In referring to graph 600, several resonant modes can be shown as peaks in the graph. The inventors have found that the resonant frequency associated with the resonant modes are related to certain critical dimensions. This shows that by producing a resonant frequency, therapeutic vibrations may be generated through sinus cavities. The resonant modes are optimal in providing the therapy disclosed herein.

Now referring to FIGS. 7(a)-8(c), one exemplary method is shown to determine an acoustic resonance for a user. The method discussed in these figures is one exemplary method, and as discussed further below, the aspects disclosed herein may be employed in determining an acoustic resonance 231 to be implemented along with the system/methods discussed herein.

Specifically, the inventors have found that the resonant frequency associated with the resonant modes are related to certain critical dimensions, described in FIGS. 7(a)-(d). The transformation from the critical dimensions (or crano-facial points) is described via equations 1-5 below.

The inventors, through experiments performed on patients have shown that when the resonant frequency, as derived from the critical dimensions discussed in FIGS. 7(a)-(d), produces therapeutic effects. The resonant modes are optimal in providing the therapy disclosed herein.

The inventors have discovered several methods of determining a resonant frequency through the measurement of critical crano-facial measurements. Once a resonant frequency is determined, it may be translated to an acoustic resonance 231, used to drive an acoustic resonance actuator 220 according to the exemplary devices disclosed herein.

In FIG. 7(a), a head 700 is shown. Three points are defined, an eye edge 710, a nostril edge 720, and a nasal midpoint 730. The distance between the eye edge 710 and the nostril edge 720, is defined as data point 1 740. The distance between the nostril edge 720 and the nasal midpoint 730, is defined as data point 2 750.

Referring to FIG. 7(b), a different view of head 700 is shown. In this view the generation of data point 3 760 is shown, which is defined by the top portion of the nose 770 and the top of the teeth 780.

To ensure the accuracy of these measurements, in an exemplary embodiment the measurements should be co-planar.

In FIG. 7(c), the mouth portion of head 700 is shown in an open state, illustrating the obtaining of a fourth data point 4 703. As shown, data point 4 770 may be defined by the middle back of the front teeth 701 to the farthest point of the hard/upper palate 702.

Referring now to FIG. 7(d), two additional data points are introduced. As shown, data point 5 783, being defined as the distance between the lowest point of an eye socket 781 to the top of teeth 782. And data point 6 792, being defined as the end of the nose cartilage 691 to the top of the teeth 782.

As exemplarily shown in FIG. 8(a), the various data points may be entered into a table 800. As shown, each of the measurements may be taken for both a right side or a left side of a user, or both. According to the aspects disclosed herein, once at least one, some, or all of the measurements in an instance for at least one or both sides are entered, a computing device 110 (as described in FIG. 2), may generate a resonate frequency data 211 employing the formula disclosed herein. Collectively, data points 1-6 may be referred to as critical measurements. However, employing the aspects disclosed herein, an exemplary implementation may use various permutations or combinations of those measurements, along with those not discussed, and other methods to generate a resonant frequency using a formula to determine one or more resonant modes/frequencies.

Referring to FIGS. 8(b) and 8(c), a front-view and a side-view of a CT scan is shown to indicate the parameters necessary to produce a resonant frequency as employed by the various systems and methods disclosed herein. As shown, in FIG. 8(b) a length of the maxillary sinus is shown via measurement 810. As shown, in FIG. 8(c), a diameter of the maxillary sinus is shown via measurement 820.

The inventors have discovered that a relationship to generate the resonant frequency for each of the right or left maxillary sinus may be obtained by exterior measurements, either obtained by manual measurements or a photograph of a user's face.

The relationship for determining resonant frequency 211 is:

$$\text{?} \qquad [ equation 1 ]$$

$$\text{?} \text{indicates text missing or illegible when filed}$$

Where:
fo is the resonant frequency 121 in hertz;
c is the speed of sound (34.3 cm/s);
π is 22/7 (used to 8 decimal places);
d is the ostial diameter for a respective right or left maxillary sinus;
l is the ostiometeal length for a respective right or left maxillary sinus;
V is the volume of the maxillary sinus for a respective right or left maxillary sinus.

As noted above, with references to FIGS. 8(b) and 8(c), conventionally, a CT-scan is needed to at least obtain the values for the ostial distance and the ostiometeal length. However, according to an exemplary embodiment, the inventors have found that the following relationship may be used to solve for the ostiometeal length (I), ostial diameter (d), and maxillary sinus volume (V),—for a respective left and right sinus. The following relationships may be employed for the calculation of a resonant frequency:

$$maxillary\_volume = width\_weight \times datapoint 1 [ 740 ] \times height\_weight \times datapoint 5 [ 783 ] \times length\_weight \times MSL \qquad [ equation 2 ]$$

$$maxillary\_ostial \_diameter ( d ) = datapoint 2 [ 750 ] / ( ostial\_weight ) \qquad [ equation 3 ]$$

$$maxillary\_ostiameteal \_length ( I ) = MSL * ostiometeal\_weight \qquad [ equation 4 ]$$

$$MSL = length\_weight * ( datapoint 3 [ 760 ] - datapoint 6 [ 791 ] ) \qquad [ equation 5 ]$$

The embodiment described above does not utilize datapoint 5 603. The inventors have discovered while said measurement may be used, as long as all the weights are set to 1, data point 5 703 may be omitted in generating a resonant frequency 131 effective in producing therapeutic benefits according to the aspects disclosed herein.

Each of equations 2-4 are solved with the measurements discussed in FIGS. 7(a)-(d). After a value is obtained for V, d, and l−a frequency for a respective right or left sinus is obtained. In one embodiment, a single frequency may be used for the right and left sinuses. In another embodiment, a right and left resonant frequency may be solved for. Thus, at least two speakers may be situated on a right and left portion respectively (for example, via the frontal sinus), and used to drive the specific resonant frequency for each side.

Experiments have found that setting each of the weights to 1, has led to a modelling of frequency that when applied as the resonant frequency according to the various aspects disclosed herein, provides an effective therapy in dealing with therapies disclosed herein. However, by collecting exact sinus dimensions for a number of patients (at least six), and measuring the various data points 1 . . . 6, applicants using equations 2-4 can solve for weights that approximate the various V, l, and d with greater accuracy using various tools, such as machine learning, linear and polynomial regression, and any other known technique for solving variables known to one of ordinary skill in the art.

Thus, equation 1 may be solved by setting each of the “_weight” to 1, and measuring the data points 1-6.

In another non-limiting example, the other data points may be estimated by using a data base that based on the known values, estimates the unknown values. The resonant frequency data 211 discussed above may be used to determine the acoustic resonance 231 shown and described in FIGS. 2 and 3.

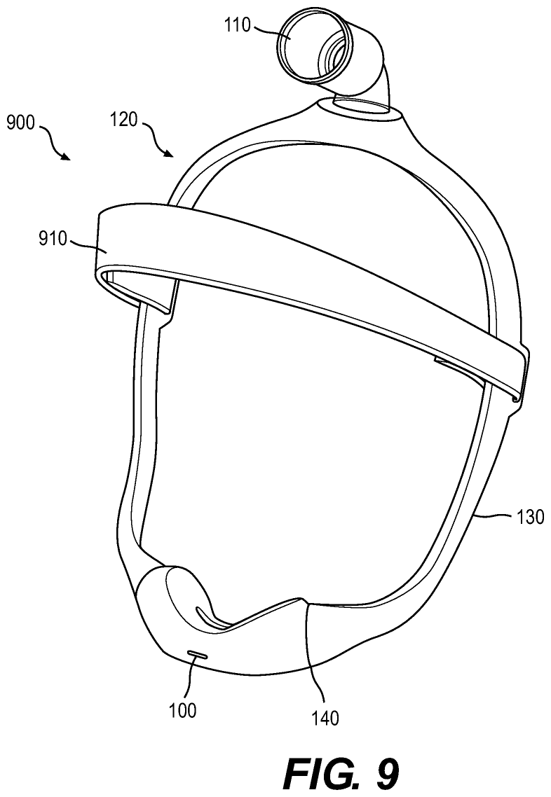

FIG. 9 illustrates a first embodiment of the aspects disclosed herein. In FIG. 9, a CPAP mask 100 as shown in FIG. 1 is provided with a band 910 (900). The band 910 is further shown in FIG. 10. As shown in FIG. 10, the band 910 includes a sound providing device 1010 (acoustic resonance actuator 220).

In one embodiment, the band 910 and the CPAP mask 100 may be integrally provided to form CPAP device 900. In another embodiment, the band 910 and the CPAP mask 100 are separately provided to form a CPAP device 900.

FIG. 10 illustrates an exemplary version of the band 910. The band 910 may be formed to fit on a user's forehead. As such, the band 910 may also be provided with fastening and adjustment techniques known in the art.

The band 910 includes a sound device 1010, that is configured to provide sound based on a methodology for generating acoustic resonance 231 (for example, as described above with FIGS. 7(a)-8(c)). The acoustic resonance 231 may be selected for the user based on a scan of their face, with the scan of their face being correlated to an acoustic resonance 231 configured specifically for that user.

Also shown in FIG. 10 are two speakers 1020 and 1030. In one exemplary embodiment, two speakers are provided, however, this example is not limiting and other numbers of speakers may be implemented. The speakers may be bone conducting speakers, as experiments have shown that bone conducting speakers are more effective in the application of acoustic resonance 231 according the implementations discussed herein. The speakers 1020 and 1030 may each be provided so as to be proximal to or directly over the frontal sinuses on the user's forehead. While the example shown in FIG. 10 is for a device providing sound on the frontal sinus, in other implementations a device may be affixed to another portion of the user's head correlating with another sinus.

As explained in FIGS. 2 and 3, the CPAP mask 100 and the band 910 (which is embodied by the acoustic resonance actuator 220), may operate integrally. Thus, when the CPAP mask 100 is configured to provide air, the band 910 may also be configured to provide an acoustic resonance 231 to the wearer, as driven by a computer device 210.

The speakers 1020 and 1030 may be slide-able as to allow for optimal placement for both comfort and effectiveness. Also shown in FIG. 10 is a microphone 1040. The microphone 1040 may be provided along with the system and method shown in FIGS. 2 and 3, to detect motion. As such, when motion is detected, both the CPAP mask 100 and band 910 may be turned on, or if configured, just the band 910 turned on to augment the CPAP mask 100. Additionally, sensors may be provided to allow for the detection of motion of a wearer (for example an accelerometer).

FIG. 11 illustrates a second embodiment according to the aspects disclosed herein. In FIG. 11, a CPAP mask 100 (for example, as shown in FIG. 1) is provided with an inlet attachment device 1100. The inlet attachment device 1100 is configured to be attached to an inlet 110.

FIG. 12 shows an inlet attachment device 1100 according to one exemplary embodiment. The inlet attachment device 1100 is a cylindrical hollow tube configured to allow air to pass from a flow generator 240 (not shown) to the CPAP mask 100 through an inlet 110.

The inlet attachment device 1100 also includes a speaker 1210, integrally attached to the housing 1201 of the inlet attachment device 1100. The speaker 1210 may be oriented so as to apply acoustic resonance 231 to the chamber 1202 of the inlet attachment device 1100.

In this way, sound generated from the speaker 1210 interacts with air going through the chamber 1202, and is delivered through the right and left passages 120 and 130 to an engagement portion 140. The acoustic resonance 231 is applied to the wearer through their nostrils.

Also shown is a sensor 1220, which may detect motion of the wearer and instigate either engagement of the speaker 1210 and/or of the CPAP mask 100.

In another example of the implementation shown in FIGS. 11 and 12, the speaker may be alternatively or additionally to, situated on the walls of the passages 120 and 130.

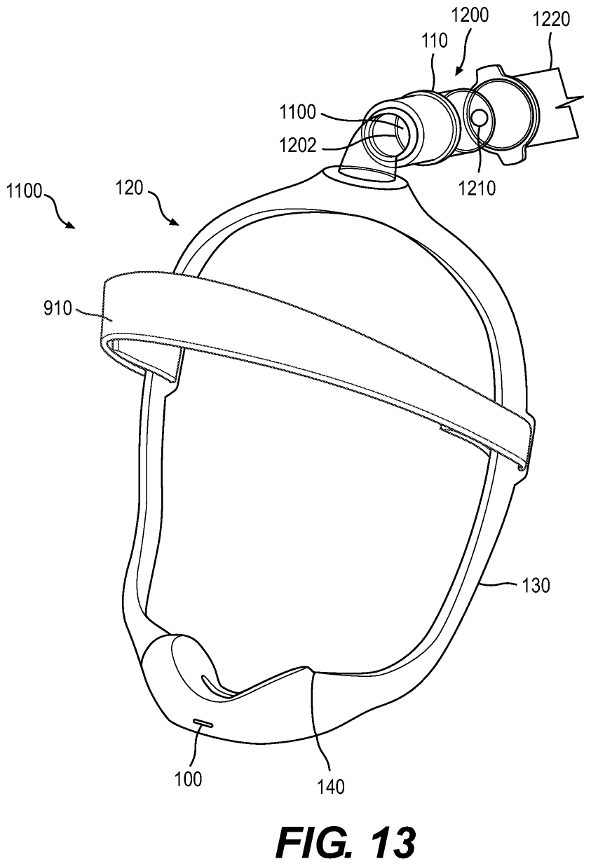

FIG. 13 illustrates a CPAP device 1300 that includes both the embodiments shown in FIGS. 9-12. As shown, the CPAP device 1300 includes a CPAP mask 100, an inlet attachment device 1100 and a band 910.

All three elements may be attached to a computing device 210 (not shown) that includes instructions as to whether to operate both the inlet attachment device 1100 and the band 910. The operation of both or each individually, may be configured and custom set based on the user's preference. Additionally, the employment of either the speaker 1210 or the sound device 1010, may be set by a feedback mechanism that gets information about the user (for example the sensors 1220 and/or microphone 1040).

In any of the implementations above, the sensors 1220 and/or the microphone 1040 may be employed to turn on/off the application of acoustic resonance 231. This may be accomplished by detecting motion or sound (for example, snoring, or tossing/turning).

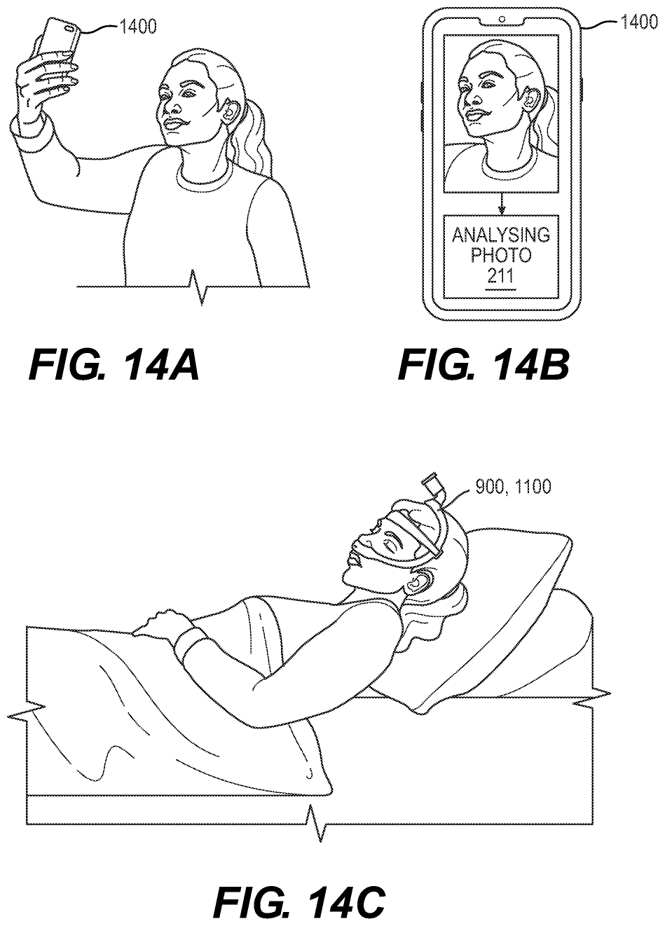

FIGS. 14(a)-(c) illustrates an example of employing the concepts disclosed herein in an application of CPAP therapy. In FIG. 14(a), a user may employ their smart phone 1400 to take a picture of their face.

As shown in FIG. 14(b) the smart phone 1400 then processes the picture of the face, for example using the methodologies described herein to create an acoustic resonance data 211. For exemplary purposes, the data transformation is shown on the smart phone 1400. However, the transformation may be done in any processing device coupled to the acoustic resonance actuator 220.

In FIG. 14(c), as the user is sleeping and wearing a device 900 or 1100, acoustic resonance data 211 is communicated to the device 900 or 1100, to an acoustic resonance actuator 220, and acoustic resonance 231 is deliver to the user while the CPAP mask 230 is employing air flow into an orifice of the user.

In the examples noted above, an acoustic resonance 231 for a specific user is calculated using a measurement of a face. This measurement of a face may be done manually by the user, or by a photograph (2D or 3D), and the acoustic resonance calculated by the extraction of critical points of the crano-facial representation of the user.

In another embodiment, various acoustic resonances may be swept, and a biometric sensor (such as those related to measuring sleep effectiveness), may be monitored. Accordingly, the acoustic resonance associated with optimal biometric sensor readings may be selected.

By employing the aspects disclosed herein, the application of acoustic resonance while delivering CPAP therapies, the application of CPAP is greatly improved, as well as compliance with wearing said device. Furthermore, a user's sleep latency may be improved (shortened), time awake after initially falling asleep, and a deep sleep by decreasing sleep fragmentation from a variety of sleep disorders.

Certain of the devices shown in FIG. 2 include a computing system (for example computing device 210). The computing system includes a processor (CPU) and a system bus that couples various system components including a system memory such as read only memory (ROM) and random access memory (RAM), to the processor. Other system memory may be available for use as well. The computing system may include more than one processor or a group or cluster of computing system networked together to provide greater processing capability. The system bus may be any of several types of bus structures including a memory bus or memory controller, a peripheral bus, and a local bus using any of a variety of bus architectures. A basic input/output (BIOS) stored in the ROM or the like, may provide basic routines that help to transfer information between elements within the computing system, such as during start-up. The computing system further includes data stores, which maintain a database according to known database management systems. The data stores may be embodied in many forms, such as a hard disk drive, a magnetic disk drive, an optical disk drive, tape drive, or another type of computer readable media which can store data that are accessible by the processor, such as magnetic cassettes, flash memory cards, digital versatile disks, cartridges, random access memories (RAMs) and, read only memory (ROM). The data stores may be connected to the system bus by a drive interface. The data stores provide nonvolatile storage of computer readable instructions, data structures, program modules and other data for the computing system.

To enable human (and in some instances, machine) user interaction, the computing system may include an input device, such as a microphone for speech and audio, a touch sensitive screen for gesture or graphical input, keyboard, mouse, motion input, and so forth. An output device can include one or more of a number of output mechanisms. In some instances, multimodal systems enable a user to provide multiple types of input to communicate with the computing system. A communications interface generally enables the computing device system to communicate with one or more other computing devices using various communication and network protocols.

The preceding disclosure refers to a number of flow charts and accompanying descriptions to illustrate the embodiments represented in FIG. 3. The disclosed devices, components, and systems contemplate using or implementing any suitable technique for performing the steps illustrated in these figures. Thus, FIG. 3 is for illustration purposes only and the described or similar steps may be performed at any appropriate time, including concurrently, individually, or in combination. In addition, many of the steps in these flow charts may take place simultaneously and/or in different orders than as shown and described. Moreover, the disclosed systems may use processes and methods with additional, fewer, and/or different steps.

Embodiments disclosed herein can be implemented in digital electronic circuitry, or in computer software, firmware, or hardware, including the herein disclosed structures and their equivalents. Some embodiments can be implemented as one or more computer programs, i.e., one or more modules of computer program instructions, encoded on a tangible computer storage medium for execution by one or more processors. A computer storage medium can be, or can be included in, a computer-readable storage device, a computer-readable storage substrate, or a random or serial access memory. The computer storage medium can also be, or can be included in, one or more separate tangible components or media such as multiple CDs, disks, or other storage devices. The computer storage medium does not include a transitory signal.

As used herein, the term processor encompasses all kinds of apparatus, devices, and machines for processing data, including by way of example a programmable processor, a computer, a system on a chip, or multiple ones, or combinations, of the foregoing. The processor can include special purpose logic circuitry, e.g., an FPGA (field programmable gate array) or an ASIC (application-specific integrated circuit). The processor also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, module, engine, software, software application, script, or code) can be written in any form of programming language, including compiled or interpreted languages, declarative or procedural languages, and the program can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

To provide for interaction with an individual, the herein disclosed embodiments can be implemented using an interactive display, such as a graphical user interface (GUI). Such GUI's may include interactive features such as pop-up or pull-down menus or lists, selection tabs, scannable features, and other features that can receive human inputs.

The computing system disclosed herein can include clients and servers. A client and server are generally remote from each other and typically interact through a communications network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

1. A system for providing positive airflow pressure (PAP), the system being couplable to a wearable PAP device, comprising:
an acoustic resonance actuator configured to deliver acoustic resonance; and
a processor configured to generate resonance data, the resonance data being wherein the acoustic resonance actuator is integrated in a band worn on the wearer's forehead. received by the acoustic resonance actuator, the resonance data corresponding to a resonant frequency based on a volume of the wearer's paranasal sinus cavity,
wherein the acoustic resonance actuator is configured to deliver the acoustic resonance when the wearable PAP device is delivering airflow,
wherein the acoustic resonance actuator is integrated in a band worn on the wearer's forehead,
the band includes bone conduction speakers configured to be worn on an area over the frontal sinus,
the resonance data is sourced from a facial scan of the wearer's face, the facial scan being defined as being sourced from a camera produced image of the wearer's face.

2-6. (canceled)

7. The system according to claim 1 further comprising a sensor.

8. The system according to claim 7, wherein the sensor detects motion or sound, and after detecting said motion or sound, applying the acoustic resonance to the wearer.

9. The system according to claim 1, wherein the volume is approximated from the facial scan.

10. A method for coupling acoustic resonance for a wearer of a positive airflow pressure (PAP) mask, comprising:
generating resonance data, the resonance data corresponding to a resonant frequency based on a volume of the wearer's paranasal sinus cavity, and
delivering the acoustic resonance to the wearer while simultaneously delivering airflow to the wearer,
wherein resonance data being sourced from a facial scan of the wearer's face,
the acoustic resonance provided to the wearer's frontal sinus,
the acoustic resonance is delivered via bone conduction speakers configured to be worn on an area over the frontal sinus,
the resonance data is sourced from a facial scan of the wearer's face, the facial scan being defined as being sourced from a camera produced image of the wearer's face.

11-15. (canceled)

16. The method according to claim 10, further comprises:
sensing an activity of the wearer.

17. The method according to claim 16, wherein the sensing is further defined as detecting motion or sound, and after detecting said motion or sound, applying the acoustic resonance to the wearer.

18. The method according to claim 10, wherein the volume is approximated from a photograph of the wearer.

19-20. (canceled)

21. The system according to claim 1, wherein the camera produced image is analyzed to produce at least two crano-facial points, the at least two crano-facial points being used to estimate the volume.

22. The method according to claim 10, wherein the camera produced image is analyzed to produce at least two crano-facial points, the at least two crano-facial points being used to estimate the volume.
